(19) United States
(12) Patent Application Publication
(10) Pub. No.: US 2012/0135952 A1
(43) Pub. Date: May 31, 2012

(54) BUTYRIC ACID SALT OF N,N-DIMETHYL IMIDOCARBON IMIDIC DIAMIDE, METHOD OF PREPARING SAME, AND PHARMACEUTICAL COMPOSITIONS AND COMBINATIONS CONTAINING SAME

(75) Inventors: Sung Wuk Kim, Seongnam-si (KR); Sung Soo Jun, Seongnam-si (KR); Changhee Min, Seoul (KR); Min Seok Kang, Goyang-si (KR); Yong Eun Kim, Daejon (KR); Ja Seong Koo, Daejon (KR)

(73) Assignee: Hanall Biopharma Co., Ltd., Daejon (KR)

(21) Appl. No.: 13/384,218

(22) PCT Filed: Jul. 16, 2010
(86) PCT No.: PCT/KR2010/004674

§ 371 Date: Feb. 10, 2012

(30) Foreign Application Priority Data
Jul. 17, 2009 (KR) 10-2009-0065254

(51) Int. Cl. A61K 31/155; C07C 277/00; A61P 35/00; A61K 31/7068; A61P 3/06; A61P 19/08; A61P 9/00; A61P 3/10; C07C 279/26; A61P 9/12

(52) U.S. Cl. 514/49; 562/606; 514/635

(57) ABSTRACT

The present invention provides metformin butyric acid salt, a method of preparing the same, and pharmaceutical compositions and combinations containing the same. The metformin butyric acid salt according to the present invention has an excellent pharmacological effect as compared with metformin hydrochloride and is capable of achieving a therapeutic purpose by administering an amount less than metformin hydrochloride. Furthermore, the metformin butyric acid salt has excellent physicochemical properties, such as solubility, stability, hygroscopicity and adsorption preventing property, in processibility of formulations and thereby is capable of being usefully utilized as a pharmaceutically acceptable salt of the metformin.

### TECHNICAL FIELD

The present invention relates to an N,N-dimethyl imidodicarbonimidic diamide butyrate, a method for preparing the same, a pharmaceutical composition containing the same, and a combination formulation containing the same.

### BACKGROUND

N,N-dimethyl imidodicarbonimidic diamide, the generic name of which is metformin, is a biguanide drug that is excellent among oral antidiabetic drugs, in terms of hypoglycemic effect and prevention of the occurrence and worsening of diabetic complications when administered as a therapeutic agent for non-insulin dependent diabetes mellitus.

It has been suggested in numerous articles that only metformin among oral antidiabetic drugs has properties as a first-line drug. In particular, it has been demonstrated that metformin activates AMP-activated protein kinase (AMPK), thus supporting the clinical validity thereof (Monica Buzzai, et al., Systemic Treatment with the Antidiabetic Drug Metformin Selectively Impairs p53-Deficient Tumor Cell Growth, Cancer Res 2007; 67:(14). July 15, 2007).

Further, it has been found that when metformin is administered to a p53 gene-deficient cancer patient, the treatment of metformin leads to changes in the energy metabolic pathway of cancer cells and an anticancer activity increases proportionally to a dose of metformin, thus showing that metformin is effective for the treatment of cancer at a normal dose for the treatment of diabetes mellitus.

According to recently published scientific articles regarding metformin, Josie M M Evans has reported that type II diabetic patients receiving metformin therapy exhibit lower carcinogenic incidence as compared to patients with no medication of metformin (Josie M M, Evans; Louise A, Donnelly; Alistair M, Emslie-Smith; Dario R, Alessi; Andrew D, Morris, BMJ. 2005, 330, 1304-1305), and Samantha L. Bowker has reported that type II diabetic patients with administration of metformin exhibit lower cancer-related mortality, as compared to patients receiving sulfonylurea or insulin therapy (Samantha L, Bowker; Sumit R, Majumdar; Paul, Veugelers; Jeffrey A, Johnson, Diabetes Care. 2006, 29, 254-258).

The free base form of metformin is pharmaceutically useful, but has low stability. For this reason, metformin is administered in the form of a pharmaceutically acceptable salt. Korean Patent No. 90,479 discloses that a pharmaceutically acceptable salt should satisfy the following physicochemical criteria: (1) good solubility; (2) good stability; (3) non-hygroscopicity; (4) anti-adhesive properties; and (5) formulation processability.

Although research relating to acid addition salts of metformin capable of meeting the above-specified properties has been continuously conducted, only the hydrochloride salt of metformin has been approved for use as a drug to date and has been used as a therapeutic agent for the treatment of non-insulin dependent diabetes mellitus. Unfortunately, such a metformin hydrochloride is poor in terms of physicochemical properties including solubility, stability, non-hygroscopicity, anti-adhesive properties and formulation processability, and therefore may have problems associated with deterioration of pharmacological effects upon being processed into formulations and hydrochloride-induced toxicity.

### DISCLOSURE

### Technical Problem

Therefore, an object of the present invention is to provide metformin butyrate which is excellent in solubility, stability, non-hygroscopicity and anti-adhesive properties and therefore exhibits excellent formulation processability and high therapeutic efficacy.

Another object of the present invention is to provide a method capable of easily preparing metformin butyrate with a high yield under mild conditions.

A further object of the present invention is to provide a pharmaceutical composition containing metformin butyrate which is highly effective for the prevention or treatment of at least one disease of diabetes mellitus, obesity, hypertension, hyperlipidemia, fatty liver, coronary artery disease, osteoporosis, cancer, myalgia, myocyte cytotoxicity and rhabdomyolysis.

A further object of the present invention is to provide a combination formulation containing metformin butyrate and a second drug.

### Technical Solution

The present invention provides metformin butyrate represented by formula 1.

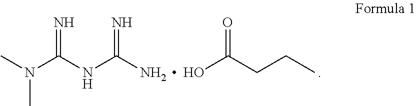

According to the present invention, the term “metformin butyrate” is intended to encompass all of metformin butyrates in the form of a crystal, an anhydride or a hydrate.

Metformin butyrate represented by formula 1 in accordance with the present invention exhibits an excellent inhibitory effect against viability and growth of cancer cells, as compared to metformin hydrochloride, and is also excellent in terms of activation of AMPKα. Accordingly, metformin butyrate represented by formula 1 exhibits superior pharmacological effects on various diseases including diabetes mellitus or cancer, as compared to metformin hydrochloride. Further, metformin butyrate represented by formula 1 has improved pharmaceutical physical properties including stability, non-hygroscopicity and formulation processability and thus metformin butyrate represented by formula 1 is more suitable for the preparation of pharmaceutical formulations as compared to metformin hydrochloride.

Further, the present invention provides a method for preparing metformin butyrate, including reacting metformin hydrochloride represented by formula 2 with a base in an organic solvent to prepare a metformin free base represented by formula 3 and reacting the metformin free base with butyric acid

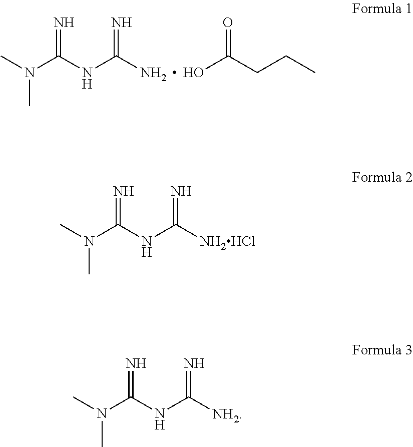

According to the present invention, a process for preparing the metformin free base has been established such that the process can be carried out simply without any special equipment. U.S. Pat. No. 4,080,472 discloses the use of an ion-exchange resin column for the removal of hydrochloride from metformin hydrochloride, or U.S. Pat. No. 4,028,402 discloses a preparation method that is carried out under severe production conditions such as the reflux of a solvent by heating and the filtration of a hot solution. However, the present invention simplifies the process in a manner that the metformin free base can be synthesized under mild conditions using general production equipment without any special equipment to enhance industrial applicability of the process. Thus, the present invention may produce the metformin free base at a lower cost. The synthesis process of the free base may also be applied to reactions with a variety of acids which have been used in the preparation of pharmaceutically acceptable salts.

According to the method of the present invention, the metformin butyrate represented by formula 1 may be prepared by Reaction Scheme 1 and Reaction Scheme 2 below:

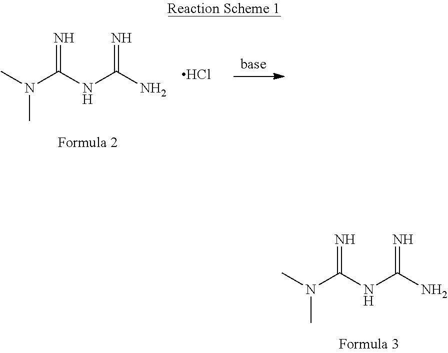

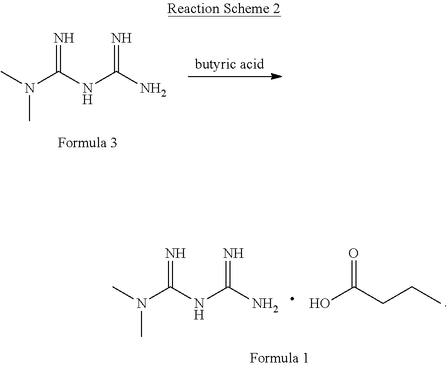

According to the reaction represented by Reaction Scheme 1, the hydrochloride of the metformin hydrochloride is removed by reacting metformin hydrochloride represented by formula 2 with a base in an organic solvent to prepare the metformin free base represented by formula 3.

The metformin free base represented by formula 3 prepared according to Reaction Scheme 1 may be a crystal form.

In the reaction represented by Reaction Scheme 1, the reaction equivalent of the base relative to metformin hydrochloride may be appropriately adjusted depending on type of base, i.e., a monovalent base, a divalent base or a trivalent base. For example, where the base is a monovalent base, the base may be used in a range of 1 equivalent to 4 equivalents, and preferably 1 equivalent to 2 equivalents, relative to 1 equivalent of metformin hydrochloride.

In the reaction represented by Reaction Scheme 1, the solvent may be an organic solvent and is preferably free of water. For example, the solvent may be methanol, ethanol, n-propanol, isopropanol, dimethyl sulfoxide (DMSO), tetrahydrofuran (THF), acetonitrile (ACN), acetone, dimethylformamide (DMF), N-methylpyrrolidone (NMP), dimethylacetamide (DMA) or a mixture thereof, and preferably isopropanol.

In the reaction represented by Reaction Scheme 1, an inorganic base used for the formation of the free base may be selected from sodium hydroxide, potassium hydroxide, calcium hydroxide, lithium hydroxide, potassium carbonate, sodium carbonate, cesium carbonate, sodium hydrogen carbonate and potassium hydrogen carbonate and may be preferably sodium hydroxide or potassium hydroxide.

According to the reaction represented by Reaction Scheme 2, the crystalline metformin butyrate represented by formula 1 may be prepared by reacting the metformin free base represented by formula 3 with butyric acid in a solvent.

In the reaction represented by Reaction Scheme 2, butyric acid is added into the metformin free base represented by formula 3 in the presence of a solvent to prepare a mixture. The mixture is stirred and then a resulting solid is filtered, washed and dried to prepare metformin butyrate represented by formula 1. Stirring may be carried out at a temperature of about −10° C. to 90° C., and preferably at a room temperature.

Metformin butyrate prepared according to Reaction Scheme 2 may be a crystal form.

In the preparation of the mixture of the metformin free base and butyric acid, 1 to 4 equivalents of butyric acid may be used relative to 1 equivalent of the metformin free base.

In the preparation of the mixture of the metformin free base and butyric acid, the solvent may be water, methanol, ethanol, n-propanol, isopropanol, dimethyl sulfoxide (DMSO), tetrahydrofuran (THF), acetonitrile (ACN), acetone, dimethylformamide (DMF), N-methylpyrrolidone (NMP), dimethylacetamide (DMA) or a mixture thereof, and preferably acetone.

Through the method for preparing metformin butyrate in accordance with the present invention, metformin butyrate may be easily prepared without using an ion exchange resin column or without involving a high-temperature heating process. Further, the reaction disclosed herein is carried out using relatively weakly acidic butyric acid and not a strong acid, such as hydrochloric acid. Therefore since metformin butyrate may be prepared under mild reaction conditions, the method of the present invention is efficient from an economic point of view and is capable of preparing metformin butyrate with high efficiency.

Further, the present invention provides a pharmaceutical composition for the prevention or treatment of a disease such as diabetes mellitus, obesity, hypertension, hyperlipidemia, fatty liver, coronary artery disease, osteoporosis, polycystic ovary syndrome, cancer, myalgia, myocyte cytotoxicity or rhabdomyolysis, containing metformin butyrate represented by formula 1

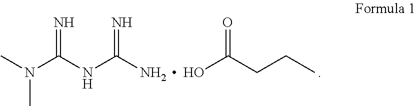

The metformin butyrate represented by formula 1 in accordance with the present invention exhibits excellent inhibitory effects against viability and growth of cancer cells, as compared to metformin hydrochloride, and is also excellent in terms of activation of AMPKα.

Metformin butyrate of formula 1 is prepared using butyric acid having less toxicity than hydrochloric acid. Also, the metformin butyrate of formula 1 exhibits a superior hypoglycemic effect to that of metformin hydrochloride. In particular, metformin butyrate of formula 1 is highly effective in lowering of a postprandial blood glucose level as well as in lowering of a fasting glucose level, and exhibits more effective inhibition of cancer cells as compared to metformin hydrochloride. As a result, metformin butyrate of formula 1 exhibits superior pharmacological effects to metformin hydrochloride, against a variety of diseases including diabetes mellitus or cancer. Therefore, a pharmaceutical composition containing metformin butyrate represented by formula 1 exhibits excellent therapeutic effects in the treatment of cancer, as well as in the treatment of diabetes mellitus, and correspondingly may be used as an anticancer drug. More specifically, the cancer may be selected from uterine cancer, breast cancer, gastric cancer, giloma, colorectal cancer, lung cancer, skin cancer, hematological cancer, liver cancer, pancreatic cancer, prostate cancer and thyroid cancer. In particular, the metformin butyrate-containing pharmaceutical composition may be very useful as an anticancer drug against breast cancer.

Further, metformin butyrate represented by formula 1 has good stability, high solubility in a variety of solvents, non-hygroscopicity and low adhesiveness, and is therefore excellent in terms of pharmaceutically required physicochemical properties including formulation processability. Consequently, upon formulation into a dosage form such as a tablet or a capsule, excellent dispersibility of metformin butyrate represented by formula 1 can lead to production of a tablet or capsule having uniform pharmacological effects, and there is no occurrence of problems associated with deterioration of pharmacological effects during the formulation process. As a result, a formulation can be produced such as a tablet or capsule having excellent therapeutic efficacy and uniform pharmacological effects.

The pharmaceutical composition containing metformin butyrate in accordance with the present invention may further include a carrier, a diluent, a binder, a disintegrant, a lubricant or other which are pharmaceutically acceptable additives.

Examples of the pharmaceutically acceptable carrier used in the present invention may include starch, microcrystalline cellulose, lactose, glucose, mannitol, light anhydrous silicic acid, alkaline earth metal salt, polyethylene glycol and dicalcium phosphate. Examples of the binder may include starch, microcrystalline cellulose, highly dispersive silica, mannitol, lactose, polyethylene glycol, polyvinylpyrrolidone, hydroxypropyl methylcellulose, hydroxypropyl cellulose, natural gum, synthetic gum, povidone, copovidone and gelatin. Examples of the lubricant include talc, light anhydrous silicic acid, stearic acid, magnesium stearate, calcium stearate, zinc stearate, lauryl sulfate, sodium lauryl sulfate, hydrogenated vegetable oil, sodium benzoate, sodium stearyl fumarate, glyceryl monostearate, polyethylene glycol 4000 and polyethylene glycol 6000.

In addition, pharmaceutically acceptable additives may be optionally used such as various additives selected from colorants and flavors.

The pharmaceutical composition containing metformin butyrate in accordance with the present invention may be preferably formulated depending on individual diseases or ingredients, by any appropriate method known in the art. For example, the aforementioned pharmaceutical composition may be formulated with further addition of a carrier, a diluent, a dispersant, a surfactant, a binder, a lubricant and various additives which are pharmaceutically acceptable.

Upon formulation of the pharmaceutical composition containing metformin butyrate in accordance with the present invention, the content of the carrier, the diluent, the dispersant, the surfactant, the binder, the lubricant and the additives to be added is not particularly limited and may be appropriately adjusted to within the content range used in conventional formulation.

The pharmaceutical composition containing metformin butyrate may be formulated in the form of a sustained-release or immediate-release tablet, a soft capsule, a hard capsule, a pill, a granule or powder, an injection or a liquid formulation, using a carrier, a diluent, a dispersant, a surfactant, a binder, a lubricant and an additive and then may be used for the prevention or treatment of pathological conditions of diabetes mellitus or its attendant diseases.

The pharmaceutical composition containing metformin butyrate in accordance with the present invention may be formulated into a sustained-release dosage form. At this time, an enteric polymer, a water-insoluble polymer, a hydrophobic compound or a hydrophilic polymer may be used as a matrix base.

As used herein, the term “enteric polymer” refers to a polymer which is insoluble or stable under acidic conditions of less than pH 5 and is dissolved or degraded under conditions of pH 5 or higher; the term “water-insoluble polymer” refers to a pharmaceutically acceptable water-insoluble polymer which controls the release of a drug; the term “hydrophobic compound” refers to a pharmaceutically acceptable water-insoluble material which controls the release of a drug; and the term “hydrophilic polymer” refers to a pharmaceutically acceptable water-soluble polymer which controls the release of a drug.

The pharmaceutical composition containing metformin butyrate in accordance with the present invention may be administered in various forms depending on desired methods, via an oral route or a parenteral route (for example, intravenously, subcutaneously, intraperitoneally or topically). Preferred may be an oral formulation.

The pharmaceutical composition of the present invention may be for use in combination with a second drug.

As used herein, the term “second drug” refers to another pharmaceutically active ingredient other than metformin butyrate of the present invention. As described above, metformin butyrate of the present invention may be used for the treatment of various diseases. Therefore, metformin butyrate of the present invention may be used in combination with the second drug for more efficient treatment of individual diseases. For example, the second drug may be an anticancer drug, an antihyperglycemic drug, an anti-obesity drug, or the like.

In one embodiment, the second drug may be an anticancer drug. The anticancer drug for use in combination with metformin butyrate represented by formula 1 may be any drug as long as it is a known anticancer drug.

Examples of the anticancer drug include known chemotherapeutic agents such as an alkylating agent, an antimetabolite, a natural product, a hormone and an antagonist and biological agents such as an immunotherapeutic agent and a gene therapeutic agent. For example, the anticancer drug may be at least one drug selected from nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate•chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temozolomide, busulfan, ifosfamide, cyclophosphamide, melphalan, altretamine, dacarbazine, thiotepa, nimustine, chlorambucil, mitolactol, leucovorin, tretinoin, exemestane, aminogluthetimide, anagrelide, navelbine, fadrozole, tamoxifen, toremifene, testolactone, anastrozole, letrozole, vorozole, bicalutamide, lomustine and carmustine.

Further, the present invention provides a combination formulation containing metformin butyrate having a structure of formula 1 and a second drug

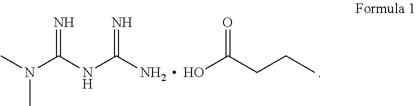

For the convenience of medication, metformin butyrate may be provided in the form of a combination formulation in which metformin butyrate in combination with a second drug is formulated into a dosage form.

In one embodiment, the second drug may be an antihyperglycemic drug, and the antihyperglycemic drug may be at least one drug selected from the group consisting of a biguanide drug, a sulfonylurea drug, a thiazolidinedione drug and an α-glucosidase inhibitor. According to another embodiment of the present invention, the second drug may be an anticancer drug, and the kind of anticancer drugs is as exemplified hereinbefore.

The dose of the pharmaceutical composition containing metformin butyrate or combination formulation in accordance with the present invention may vary depending on the patient's weight, age, gender, nationality, health status and diet, administration time, administration route, excretion rate and disease severity, and divisional administration of the pharmaceutical composition or combination formulation may be also pursuant to discretion of a physician. For example, the pharmaceutical composition containing metformin butyrate or combination formulation in accordance with the present invention may be orally administered at a frequency of once to three times a day, such that the content of the active ingredient metformin is in a range of 50 mg to 3,000 mg.

### Advantageous Effects

The crystalline metformin butyrate in accordance with the present invention exhibits superior pharmacological effects on various diseases including diabetes mellitus and cancer, as compared to metformin hydrochloride which has been conventionally used as an antidiabetic drug. In addition, metformin butyrate is excellent in terms of physicochemical properties such as solubility, stability, non-hygroscopicity and anti-adhesive properties.

Further, the method for preparing metformin butyrate in accordance with the present invention is capable of producing a novel salt of crystalline metformin at a lower cost by simplifying the production process in a manner that metformin butyrate can be synthesized in general production equipment without any special equipment, thereby enhancing industrial applicability of the process.

The pharmaceutical composition containing crystalline metformin butyrate in accordance with the present invention exhibits excellent pharmacological effects. Further, since metformin butyrate has excellent physicochemical properties, a pharmaceutical composition containing the metformin butyrate is readily formulated into a tablet or a capsule.

Further, the combination formulation containing both metformin butyrate and a second drug in accordance with the present invention has excellent pharmacological effects and is capable of achieving medication convenience of patients.

### DESCRIPTION OF THE DRAWING

FIG. 1 shows an activation degree of AMPKα by metformin butyrate in accordance with the present invention.

### DETAILED DESCRIPTION

Hereinafter, embodiments of the present invention will be described in more detail with reference to the following Examples, Formulation Examples and Experimental Examples. However, it should be understood that the following Examples are provided only for illustrating the present invention and should not be construed as limiting the scope and spirit of the present invention.

Unless otherwise specifically indicated, reagents and solvents referred to hereinafter were purchased from Aldrich (USA) and Daejung Chemicals & Metals Co., Ltd. (South Korea). ¹H-NMR and ¹³C-NMR data were measured using a Varian INOVA-600 MHz FT-NMR (Varian, USA), and the melting point (mp) was measured using an Electrothermal digital melting point apparatus No. 9201 (Electrothermal, GB).

### EXAMPLES

### Example 1

### Preparation of Metformin Free Base

16.6 g of metformin hydrochloride and 6.0 g of 93 wt % potassium hydroxide were added to 50 mL of isopropanol, followed by stirring at 50° C. for 2 hours. The reaction solution was cooled to 25° C., filtered, and then washed with 20 mL of isopropanol. Thereafter, the reaction solution was further washed once with 20 mL of acetone, concentrated, and dried under vacuum to give 12.8 g (yield: 98.5%) of a metformin free base as a white solid.

Melting point: 119.0 to 119.5° C.

¹H-NMR (600 MHz, D₂O) δ(ppm) 3.07(s, 6H)

¹³C-NMR (150 MHz, D₂O) δ(ppm) 161.05, 158.5, 37.35

### Example 2

### Preparation of Metformin Butyrate

10.0 g (1.0 equivalent) of the metformin free base prepared in Example 1 was dissolved in 150 mL of acetone. To the reaction liquid was slowly added dropwise 7.1 mL (1.2 equivalents) of butyric acid under stirring, followed by stirring at room temperature for 2 hours. The resulting solid was filtered, washed successively with 20 mL of isopropanol and 50 mL of acetone, and then dried with hot air to give 16.4 g (yield: 98.0%) of metformin butyrate as a white solid.

Melting point: 162° C.

¹H-NMR (600 MHz, D₂O) δ(ppm) 3.01(s, CH₃, 6H), 2.12(t, J=7.2 Hz, CH₂, 2H), 1.53(m, CH₂, 2H), 0.86(t, J=7.2 Hz, CH₃, 3H)

¹³C-NMR (150 MHz, D₂O) δ(ppm) 184.13, 160.18, 158.57, 37.54, 29.53, 19.49, 13.37

### Formulation Examples

### Formulation Example 1

### Preparation of Tablet Containing Metformin Butyrate

327.97 g of metformin butyrate and 61.03 g of microcrystalline cellulose were individually sieved using a No. 20 sieve and then mixed in a V-type mixer for 60 minutes. Meanwhile, 15 g of Kollidon VA64 (BASF, Germany) and 4 g of light anhydrous silicic acid were sieved using a No. 35 sieve and added to the above mixture, followed by mixing for 60 minutes. Finally, 2 g of stearic acid was sieved using a No. 35 sieve and added to the above mixture, followed by mixing for 3 minutes.

Then, the final mixture was compressed to prepare a tablet layer containing 327.97 mg of metformin butyrate/tablet, and 10 mg of a film-coated layer/tablet was formed thereon using Opadry OY-C-7000A as a coating base in a Hi-coater (SFC-30F, Sejong Pharmatech Co., Ltd., South Korea), thereby preparing a tablet containing metformin butyrate.

### Formulation Example 2

### Preparation of Tablet Containing Metformin Butyrate

655.93 g of metformin butyrate and 114.07 g of dicalcium phosphate were individually sieved using a No. 35 sieve and then mixed in a high-speed mixer for 3 minutes. Meanwhile, 20 g of povidone K-30 was added and dissolved in 100 g of ethanol to prepare a binding solution, which was then added to the high-speed mixer, followed by kneading for 3 minutes. The kneadate was dried in a steam drier and then granulated through a No. 20 sieve. 5 g of light anhydrous silicic acid was sieved using a No. 35 sieve and added to the above mixture, followed by mixing in a V-type mixer for 60 minutes. Finally, 5 g of magnesium stearate was sieved using a No. 35 sieve and added to the above mixture, followed by mixing for 3 minutes.

Then, the final mixture was compressed to prepare a tablet layer containing 655.93 mg of metformin butyrate/tablet, and 20 mg of a film-coated layer/tablet was formed thereon using Opadry OY-C-7000A as a coating base in a Hi-coater (SFC-30F, Sejong Pharmatech Co., Ltd., South Korea), thereby preparing a metformin tablet containing metformin butyrate.

### Formulation Example 3

### Preparation of Sustained-Release Tablet Containing Metformin Butyrate

327.97 g of metformin butyrate and 314.03 g of hydroxypropyl methylcellulose 2208 (viscosity: 100,000 cps) were individually sieved using a No. 20 sieve and then mixed in a double cone mixer for 60 minutes. Meanwhile, 20 g of hydroxypropyl cellulose and 4 g of light anhydrous silicic acid were sieved using a No. 35 sieve and added to the above mixture, followed by mixing for 60 minutes. Finally, 4 g of stearic acid was sieved using a No. 35 sieve and added to the above mixture, followed by mixing for 3 minutes.

Then, the final mixture was compressed to prepare a sustained-release tablet layer containing 306.58 mg of metformin butyrate/tablet, and 20 mg of a film-coated layer/tablet was formed thereon using Opadry OY-C-7000A as a coating base in a Hi-coater (SFC-30F, Sejong Pharmatech Co., Ltd., South Korea), thereby preparing a sustained-release tablet containing metformin butyrate.

### Formulation Example 4

### Preparation of Sustained-Release Tablet Containing Metformin Butyrate

327.97 g of metformin butyrate, 44.03 g of microcrystalline cellulose, and 300 g of polyethylene oxide (molecular weight: 5,000,000, Dow Chemical Company Ltd., USA) were sieved using a No. 20 sieve and then mixed in a double cone mixer for 60 minutes. Meanwhile, 20 g of Kollidon VA64 and 4 g of light anhydrous silicic acid were sieved using a No. 35 sieve and added to the above mixture, followed by mixing for 60 minutes. Finally, 4 g of stearic acid was sieved using a No. 35 sieve and added to the above mixture, followed by mixing for 3 minutes.

Then, the final mixture was compressed to prepare a sustained-release tablet layer containing 306.58 mg of metformin butyrate/tablet, and 20 mg of a film-coated layer/tablet was formed thereon using Opadry OY-C-7000A as a coating base in a Hi-coater (SFC-30F, Sejong Pharmatech Co., Ltd., South Korea), thereby preparing a sustained-release tablet containing metformin butyrate.

### Formulation Example 5

### Preparation of Capsule Containing Metformin Butyrate

163.98 g of metformin butyrate and 53.52 g of microcrystalline cellulose were individually sieved using a No. 20 sieve and then mixed in a V-type mixer for 60 minutes. 1.5 g of light anhydrous silicic acid was sieved using a No. 35 sieve and added to the above mixture, followed by mixing for 60 minutes. Finally, 1 g of stearic acid was sieved using a No. 35 sieve and added to the above mixture, followed by mixing for 3 minutes.

Then, the final mixture was filled into a blank capsule to prepare a capsule containing 153.39 mg of metformin butyrate/capsule.

### Formulation Example 6

### Preparation of Tablet Containing Metformin Butyrate and Capecitabine

327.97 g of metformin butyrate, 75 g of capecitabine, 105.03 g of microcrystalline cellulose and 50 g of dicalcium phosphate were individually sieved using a No. 20 sieve and then mixed in a V-type mixer for 60 minutes. Meanwhile, 15 g of Kollidon VA64 (BASF, Germany) and 4 g of light anhydrous silicic acid were sieved using a No. 35 sieve and added to the above mixture, followed by mixing for 60 minutes. Finally, 3 g of stearic acid was sieved using a No. 35 sieve and added to the above mixture, followed by mixing for 3 minutes.

Then, the final mixture was compressed to prepare a tablet layer containing 327.97 mg of metformin butyrate and 75 mg of capecitabine/tablet, and 20 mg of a film-coated layer/tablet was formed thereon using Opadry OY-C-7000A as a coating base in a Hi-coater (SFC-30F, Sejong Pharmatech Co., Ltd., South Korea), thereby preparing a tablet containing metformin butyrate and capecitabine.

### Experimental Examples

### Experimental Example 1

### Growth-Inhibitory Effects of Metformin Butyrate on Cancer Cells

Metformin butyrate synthesized according to the method described in Example 2 of the present invention was applied to cancer cells to measure cancer cell growth-inhibitory effects of metformin butyrate. A brief experimental method is as follows.

Human breast cancer-derived MCF7 cells and lung cancer-derived A549 cells were used as a test system. A cellular viability (%) and a concentration of metformin butyrate which provides 50% inhibition of cell growth (growth inhibitory concentration, GIC50) were measured using an MTT (3-(4,5-dimethylthiazol-2-yl)-2,5-ditetrazoliumbromide) assay to confirm the cancer cell growth-inhibitory effect of metformin butyrate.

MCF7 cells and A549 cells cultured in a DMEM containing 10% fetal bovine serum were respectively dispensed at a cell density of about 5×10³ cells/well on a 96-well plate, followed by culturing for about 24 hours. In order to examine cellular viability, 2 mM or 10 mM of metformin butyrate prepared in Example 2 was applied to the culture media, followed by culturing for 72 hours. In order to calculate a GIC50 value, 10 mM, 2 mM, 0.4 mM, 0.08 mM and 0.016 mM of metformin butyrate were respectively applied to the culture media, followed by culturing for 72 hours.

In order to confirm viable cells after treatment of metformin butyrate, MIT was added to the culture media, followed by culturing for another 3 hours. The resulting formazane crystal was dissolved in dimethyl sulfoxide (DMSO), and absorbance of the solution was measured at 560 nm.

The ratio of the count of viable cells in the well plate with application of metformin butyrate relative to the count of cells cultured in the well plate with no treatment of metformin butyrate after culturing for 72 hours is expressed in terms of cellular viability (%). In addition, using a cellular viability curve, the concentration value of metformin butyrate providing 50% inhibition of cell growth (GIC50) was calculated to confirm cancer cell growth-inhibitory effects of metformin butyrate. The results are given in Tables 1 and 2 below, respectively.

Further, cellular viability (%) and GIC50 values were calculated using metformin hydrochloride or butyric acid instead of metformin butyrate. The results are given in Tables 1 and 2 below, respectively.

TABLE 1
CELLULAR VIABILITY (%)

| | |
|---|---|
| MCF7 | A549 |

| Ingredient | 2 mM | 10 mM | 2 mM | 10 mM |
|---|---|---|---|---|
| Metformin | 45.5% | 19.9% | 57.7% | 11.7% |
| butyrate | | | | |
| Metformin | 86.5% | 73.6% | 63.5% | 39.6% |
| hydrochloride | | | | |
| Butyric acid | 53.9% | 22.3% | 78.6% | 28.3% |

TABLE 2
GROWTH INHIBITORY CONCENTRATION (GIC50)

| Ingredient | MCF7 | A549 |
|---|---|---|
| Metformin butyrate | 1.6 mM | 2.5 mM |
| Metformin hydrochloride | >10 mM | 3.6 mM |
| Butyric acid | 2.2 mM | 5.2 mM |

As seen from the cellular viability of Table 1, Metformin butyrate exhibited a lower cell viability than metformin hydrochloride with respect to MCF7 and A549. From this result, it may be seen that metformin butyrate shows more effective inhibition of cancer cell than metformin hydrochloride. In particular, when MCF7 and A549 cells were cultured in the presence of 10 mM metformin butyrate, a cellular viability was less than 20%. From this result, it may be seen that metformin butyrate is capable of effectively inhibiting viability of breast cancer and lung cancer cells.

On the other hand, as seen from GIC50 values of Table 2, metformin butyrate exhibited effective inhibition of the growth of MCF7 and A549 cells at a much lower dose than that of metformin hydrochloride. From this result, it can be seen that metformin butyrate is capable of effectively inhibiting the growth of cancer cells derived from breast cancer and lung cancer, at a lower dose than that of metformin hydrochloride.

### Experimental Example 2

### Effects of Metformin Butyrate on Activation of AMPKα

Metformin butyrate synthesized according to the method described in Example 2 of the present invention and metformin hydrochloride were individually applied to cells to measure effects of the activation of 5′-AMP-activated protein kinase alpha (AMPKα). A brief experimental method is as follows.

Human breast cancer-derived MCF7 cells were used as a test system. Effect of metformin butyrate on the activation of AMPKα was confirmed using an AMPKα immunoassay kit (Catalog No. KH00651, Invitrogen).

MCF7 cells were cultured in a DMEM containing 10% fetal bovine serum and dispensed at a cell density of about 5×10⁵ cells/well on a 6-well plate, followed by cell culture in a 5% CO₂ incubator. 0.4 mM, 2 mM and 10 mM of metformin butyrate were respectively treated to the culture media, and the cells were incubated for 24 hours.

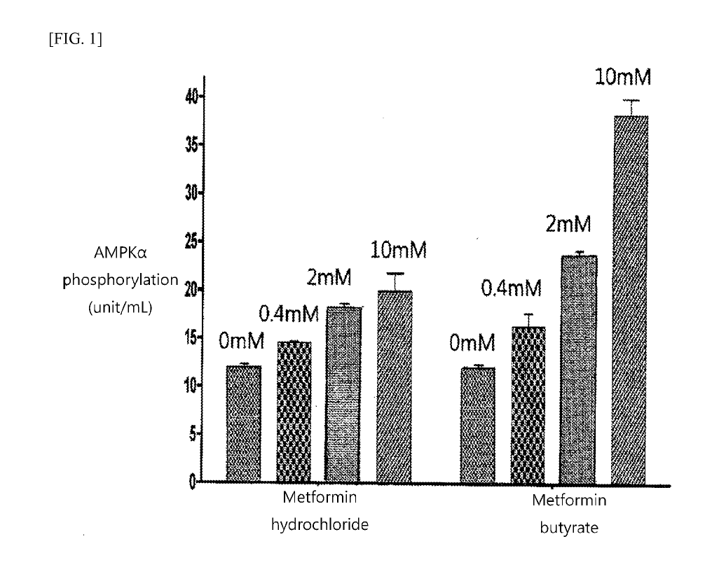

The phosphorylation of threonine residue 172 (T172) of AMPKα in cells cultured in the presence of metformin butyrate and cells cultured in the absence of metformin butyrate as negative control was confirmed using an AMPKα immunoassay kit (Catalog No. KHO0651, Invitrogen). The cells were lysed according to the instructions involved in the AMPKα immunoassay kit (Catalog No. KHO0651, Invitrogen). After protein quantification of cellular lysate, the phosphorylation degree of AMPKα T172 was confirmed from the 20 μg of cellular lysate using the method described in the instructions involved in the AMPKα immunoassay kit. The results are given in Table 3 below and FIG. 1. The ratio of T172-phosphorylation of the cells cultured in the metformin butyrate-containing media relative to T172-phosphorylation of the cells cultured in the metformin butyrate-free media is given in Table 3 below.

Further, the phosphorylation of AMPKα T172 was examined substantially in the same manner as above, using metformin hydrochloride instead of metformin butyrate. The results are given in Table 3 below.

TABLE 3

| | | 0 mM | 0.4 mM | 2 mM | 10 mM |
|---|---|---|---|---|---|
| Metformin | Value | 11.9 ± | 16.3 ± | 23.8 ± | 38.3 ± |
| butyrate | measured | 0.50 | 1.98 | 0.64 | 1.2 |
| | | unit/mL | unit/mL | unit/mL | unit/mL |
| | Ratio | 1.0 | 1.4 | 2.0 | 3.2 |
| Metformin | Value | 11.9 ± | 14.5 ± | 18.2 ± | 19.95 ± |
| hydrochloride | measured | 0.50 | 0.21 | 0.70 | 2.6 |
| | | unit/mL | unit/mL | unit/mL | unit/mL |
| | Ratio | 1.0 | 1.2 | 1.5 | 1.7 |

As seen from Table 3 and FIG. 1, metformin butyrate exhibited higher phosphorylation of AMPKα T172 than metformin hydrochloride at the same concentration. Accordingly, it can be seen that metformin butyrate contributes to more efficient activation of AMPKα than metformin hydrochloride, and consequently a pharmaceutical composition containing metformin butyrate is capable of exhibiting excellent effects against diseases such as diabetes mellitus, obesity, hypertension, hyperlipidemia, fatty liver, coronary artery disease, osteoporosis or polycystic ovary syndrome, cancer, myalgia, myocyte cytotoxicity and rhabdomyolysis.

### INDUSTRIAL APPLICABILITY

Metformin butyrate of the present invention has an excellent pharmacological effect as compared to metformin hydrochloride and is capable of achieving a therapeutic purpose at a dose lower than that of metformin hydrochloride. Further, metformin butyrate has excellent physicochemical properties in terms of formulation processability, such as solubility, stability, hygroscopicity and anti-adhesive properties, and is therefore useful as a pharmaceutically acceptable salt of metformin. Further, metformin butyrate may be provided in the form of a combination formulation, such that metformin butyrate can be administered in combination with an additional drug.

1. Metformin butyrate represented by formula 1:

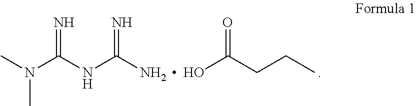

2. The metformin butyrate of claim 1, wherein the metformin butyrate is in the form of a crystal.

3. The metformin butyrate of claim 2, wherein the crystalline metformin butyrate has a melting point of 162° C.

4. A method for preparing metformin butyrate represented by formula 1, comprising:
reacting metformin hydrochloride represented by formula 2 with a base in an organic solvent to prepare a metformin free base represented by formula 3; and
reacting the metformin free base with a butyric acid, wherein formulae 1, 2 and 3 are represented by:

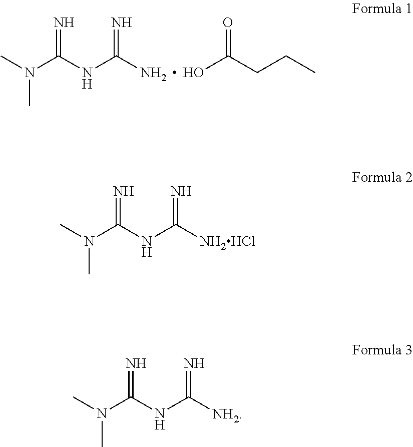

5. The method of claim 4, wherein reacting the metformin free base with butyric acid comprises:
adding the butyric acid to the metformin free base represented by formula 3 to prepare a mixture; and
stirring the mixture to prepare a solid, and then filtering, washing and drying the solid.

6. A pharmaceutical composition for the prevention or treatment of at least one disease selected from diabetes mellitus, obesity, hypertension, hyperlipidemia, fatty liver, coronary artery disease, osteoporosis, polycystic ovary syndrome, cancer, myalgia, myocyte cytotoxicity and rhabdomyolysis, comprising a pharmaceutically acceptable carrier, diluent, dispersant, surfactant, binder or lubricant, in combination with metformin butyrate represented by formula 1:

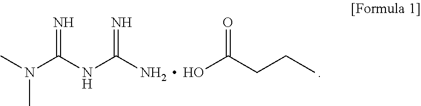

7. The pharmaceutical composition of claim 6, wherein the cancer is selected from uterine cancer, breast cancer, gastric cancer, giloma, colorectal cancer, lung cancer, skin cancer, hematological cancer, liver cancer, pancreatic cancer, prostate cancer and thyroid cancer.

8. The pharmaceutical composition of claim 7, wherein the cancer is at least one selected from breast cancer and lung cancer.

9. The pharmaceutical composition of claim 6, wherein the pharmaceutical composition is used as an anticancer drug.

10. The pharmaceutical composition of claim 6, wherein the pharmaceutical composition is formulated in a tablet, a capsule, a pill, a granule, a powder, a sterile injection or a liquid formulation.

11. The pharmaceutical composition of claim 6, wherein the pharmaceutical composition is formulated in a sustained-release tablet.

12. The pharmaceutical composition of claim 6, wherein the pharmaceutical composition is for use in combination with a second drug.

13. The pharmaceutical composition of claim 12, wherein the second drug is an anticancer drug.

14. The pharmaceutical composition of claim 13, wherein the anticancer drug is at least one selected from nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate•chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temozolomide, busulfan, ifosfamide, cyclophosphamide, melphalan, altretamine, dacarbazine, thiotepa, nimustine, chlorambucil, mitolactol, leucovorin, tretinoin, exemestane, aminogluthetimide, anagrelide, navelbine, fadrozole, tamoxifen, toremifene, testolactone, anastrozole, letrozole, vorozole, bicalutamide, lomustine and carmustine.

15. The pharmaceutical composition of claim 6, wherein the pharmaceutical composition is for the prevention or treatment of both cancer and diabetes mellitus.

16. A combination formulation comprising metformin butyrate represented by formula 1 and a second drug

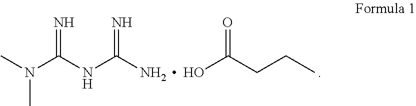

17. The combination formulation of claim 16, wherein the second drug is an anticancer drug.

18. The combination formulation of claim 17, wherein the anticancer drug is at least one drug selected from nitrogen mustard, imatinib, oxaliplatin, rituximab, erlotinib, neratinib, lapatinib, gefitinib, vandetanib, nilotinib, semaxanib, bosutinib, axitinib, cediranib, lestaurtinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate•chitosan, gemcitabine, doxifluridine, pemetrexed, tegafur, capecitabine, gimeracil, oteracil, azacitidine, methotrexate, uracil, cytarabine, fluorouracil, fludarabine, enocitabine, flutamide, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temozolomide, busulfan, ifosfamide, cyclophosphamide, melphalan, altretamine, dacarbazine, thiotepa, nimustine, chlorambucil, mitolactol, leucovorin, tretinoin, exemestane, aminogluthetimide, anagrelide, navelbine, fadrozole, tamoxifen, toremifene, testolactone, anastrozole, letrozole, vorozole, bicalutamide, lomustine and carmustine.
